(19) United States
(12) Patent Application Publication
(10) Pub. No.: US 2020/0024561 A1
(43) Pub. Date: Jan. 23, 2020

(54) DEVICE FOR CELL CULTURE AND METHOD FOR CULTURING CELLS

(71) Applicant: AGENCY FOR SCIENCE, TECHNOLOGY AND RESEARCH, Singapore (SG)

(72) Inventors: May WIN NAING, Singapore (SG); Dan LIU, Singapore (SG); Abdul Rahim AHMAD AMIRUL, Singapore (SG); Deepak CHOUDHURY, Singapore (SG)

(73) Assignee: AGENCY FOR SCIENCE, TECHNOLOGY AND RESEARCH, Singapore (SG)

(21) Appl. No.: 16/497,156

(22) PCT Filed: Mar. 29, 2018
(86) PCT No.: PCT/SG2018/050162

§ 371 Date: Sep. 24, 2019

(30) Foreign Application Priority Data
Mar. 30, 2017 (SG) 10201702629Q

(51) Int. Cl. C12M 1/24; C12M 1/00; C12M 1/34

(52) U.S. Cl. CPC C12M 23/08; C12M 41/44; C12M 23/50; C12M 23/34

(57) ABSTRACT

Disclosed is a device for cell culture, the device includes a first chamber having an area for containing cells, such as isolated cells, for growth; a second chamber for containing culture medium; a semi permeable membrane forming at least part of a divider between the first chamber and the second chamber; wherein the area for containing cells for growth is adjustable via a moveable wall disposed within the first chamber. The device is especially suited for culturing of semi-adherent and non-adherent cells.

### TECHNICAL FIELD

The present invention relates to a device for cell culture and method for culturing cells. The invention is suited for, but not limited to culturing of semi-adherent and non-adherent cells.

### BACKGROUND

Cells and tissues are cultured for a variety of applications, such as for general cell therapy, immunotherapy, biopharmaceutical process development and general research.

Conventional devices for culturing cells typically include T-flasks and well plates. In a conventional process such as a 24-well plate arrangement, cells are transferred and medium is changed well by well. As cell numbers increase, cells are passaged and transferred from one well plate/flask to multiple plates/flasks. This manual process is very time consuming, susceptible to contamination and highly dependent on manual labor.

Another commonly used device for cell culture is spinner flasks, which are especially designed for suspension cultures and may allow better gas exchange than T-flasks and well plates. Spinner flasks work on the basis of aeration of cultures afforded by a stirrer. In order for the cell culture to have proper aeration, the total culture volume in a spinner flask should not exceed half of the indicated volume of the flask.

For dedicated cell or tissue culture applications, specialised devices such as bioreactors are developed to maintain optimal or near optimal conditions or parameters suitable for cell growth/expansion. Such parameters may include maintenance of oxygen at a certain level, providing nutrient supply through flow control, and/or increased surface area for scale up.

However, current devices/bioreactors face one or more of the following problems. Firstly, certain type of cells such as antigen-specific immune cells have a specific requirement for culture density which allows the cells to interact with each other and grow at an optimum rate. Current cell expansion systems require cells to be seeded and maintained within the same culture vessel (with fixed volume), leading to lower than optimal cell density at the beginning of the culture but higher than optimal cell density at the later stage of the culture.

Further, when cells become too dense, the growth rate of the cells will slow down and eventually plateau. It is pertinent to bring down the cell density, through expanding of culture area/volume, as well as introduction of additional culture medium. To achieve this, cells are typically transferred to a new vessel of a larger volume or multiple new vessels of the same size. The process of transferring involves multiple steps such as harvesting the cells, centrifuging the cells, decanting the old medium, suspending or re-suspending cells with a new medium and reallocating cells into the new vessel(s). It is appreciable that each of the steps could increase the amount of time required and introduce contamination risk to the cell culture.

Besides contamination, some cells, especially antigen-specific T cells are sensitive to movement caused by medium stirring or wave motion. Most of the current systems introduce stirring or rocking motions, which are thus non-ideal for culturing such cells which are susceptive to flow shear.

Cultured cells secrete substances such as cytokines and other proteins into their environment which can facilitate further cell expansion and growth. With current systems, essential cell secreted proteins are diluted and lost to different degrees in the cell environment during the steps of cell transfer to multiple culture vessel(s). Further, during steps of cell transfer or medium change, cell growth may inadvertently be interrupted.

It is well established that gaseous exchange (oxygen and carbon dioxide) is important for cell growth. There are two methods to achieve gaseous exchange in conventional systems. One method is by oxygenizing the medium and agitating the medium to homogenize gas distribution in a bulk liquid medium. This is non-ideal because it introduces agitation which can be bad for some types of cells. In addition, the step of oxygenation of medium requires special equipment which may increase overheads/costs. Another method as employed by in G-Rex® bioreactors is to culture the cells on top of one or more gas permeable membranes to facilitate direct gaseous supply to the cells. However, the gas permeability of the membrane may be compromised with the expansion of cells which sediment under gravity and cover the membrane with (for semi-adherent cells) or without attachment (for suspension cells).

Conventional methods for semi and non-adherent cells include cell harvesting, cell centrifugation, decanting of old medium, and resuspension of cells in fresh medium. These steps are manual labor intensive and the accurate execution may depend on the skills of such manual labor.

In addition to the aforementioned problems, there exists a need to reduce the dependence of manual labor and time for culturing cells. In particular, there exist a need to support continuous cell culture with reduced/minimized cell growth disruption.

It is an object of the invention to provide improved devices to alleviate one or more of the aforementioned problems for a variety of cell types.

### SUMMARY OF THE INVENTION

The invention was conceptualized to tackle problems with current cell culture systems for semi-adherent and non-adherent cells, particularly, but not limited to antigen specific immune cells. These problems are related to culturing and maintaining of cells at their optimum density, contamination risks from multiple handling steps, and non-ideal conditions for culturing shear sensitive cells.

According to an aspect of the invention there is a device for cell culture comprising a first chamber defining an area for containing isolated cells for growth; a second chamber for containing culture medium; a semi permeable membrane forming at least a portion of a divider between the first chamber and the second chamber; wherein the area (and appreciably an enclosed volume defined by the area) for containing isolated cells for growth is adjustable.

The device according to the disclosure advantageously allows generic cell culture steps can be performed within a single vessel and cells can be expanded over long periods of time without the need to transfer these cells across different culture vessels. In addition, by keeping the second chamber separated from the first chamber, culture medium may be replaced/introduced without causing undue stress to shear sensitive cells.

In some embodiments, the semi-permeable membrane comprises uniform micro-sized pores, the diameter of each micro-sized pore is between 0.1 to 5 μm.

In some embodiments, the semi-permeable membrane is supported by a grid.

In some embodiments, the first chamber comprises a port shaped and dimensioned to receive a piston therein. The piston may be arranged to define the area for containing isolated cells for growth by movement along an axis of the first chamber. In some embodiments, the axis may be a longitudinal axis. In other embodiments, there area may be adjusted via one or more other axes. The port may be coated with a sealant such as rubber.

In some embodiments, the piston is coated with a sealant. The sealant may be rubber, in particular a bio-compatible rubber such as polydimethylsiloxane.

According to another aspect of the invention there is a method for cell culture, including the steps of: providing a first chamber, the first chamber having an area for containing isolated cells for growth; providing a second chamber for containing culture medium; dividing the first chamber and the second chamber using a semi permeable membrane, the semi permeable membrane forming part of a divider between the first chamber and the second chamber; wherein the area for containing isolated cells for growth is adjustable via a moveable wall disposed within the first chamber.

According to another aspect of the invention there is a system for cell culture comprising the device as mentioned; and, a control mechanism; wherein the control mechanism is operable to adjust the area for containing isolated cells for growth via moving the moveable wall.

According to another aspect of the invention there is a device for cell culture, the device includes a first chamber having an area for containing cells for growth and culture medium; wherein the area is adjustable via a moveable wall disposed within the first chamber; and wherein the device is arranged to form a closed environment such that the area is isolated from the ambient environment.

The invention enables in-situ culture area expansion and optimum cell density regulation without the need of transferring cells to larger/multiple vessels, and/or partial change of medium through the medium chamber without the need for conventional non-adherent culture medium change procedures. This reduces user-handling, cell exposure to the ambient environment, and therefore achieves reduction in total contamination risk.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures, which illustrate, by way of example only, embodiments of the present invention,

FIG. 1a shows a perspective view of a device for cell culture;

FIG. 1b shows a cross-section view of the device for cell culture;

FIG. 2 shows a cross-section view of the device in use with cell chamber on top, and medium chamber at the bottom, the first chamber and second chamber separated by a semi-permeable membrane, cells inside the culture region are presented as granules;

FIG. 3 shows a cross-section view of the device in use with cell culture area expanded relative to FIG. 2, cells inside the culture region are presented as granules;

FIG. 4a and FIG. 4b shows the corresponding top views of the device of FIG. 2 and FIG. 3 respectively;

FIG. 5 shows the cell growth comparison between the device of the invention and 24-well plate device as control.

### DESCRIPTION OF EMBODIMENTS

Throughout the description, the term ‘cell’ and its plural form may include, and is not limited to “mammalian cell”, “plant cell” and “bacterial cell”.

Throughout the description, the term ‘area’ broadly covers a two-dimensional aspect of a space. The term ‘volume’ is construed as a three-dimensional aspect of an enclosed space.

Throughout the description, unless otherwise stated, the terms ‘medium’ and ‘culture medium’ are used interchangeably.

In accordance with an aspect, there is a device for cell culture, the device includes a first chamber having an area for containing cells, such as isolated cells for growth; a second chamber for containing culture medium; a semi permeable membrane forming at least part of a divider between the first chamber and the second chamber; wherein the area for containing isolated cells for growth is adjustable via a moveable wall disposed within the first chamber.

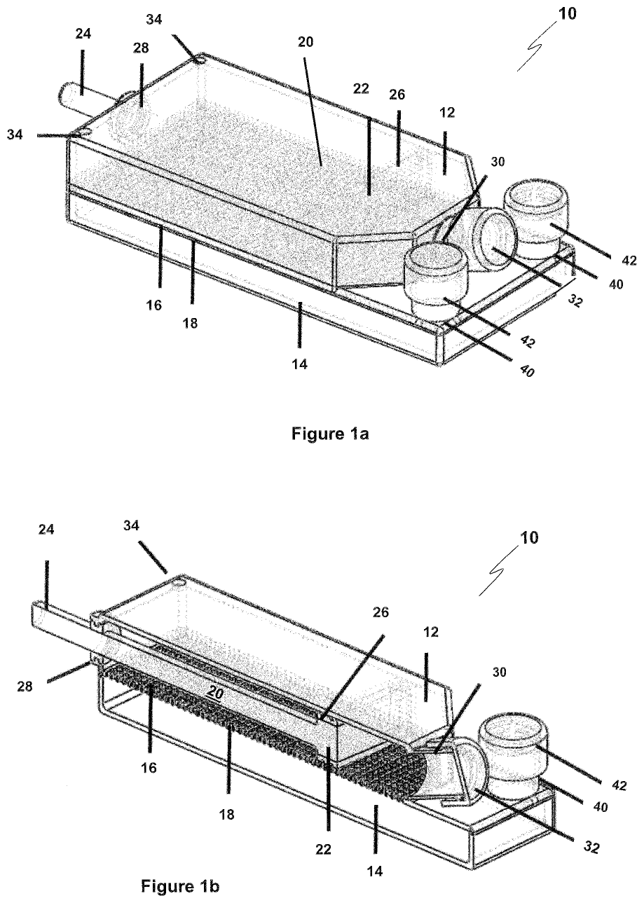

With reference to the FIGS. 1 to 4, the device 10 for cell culture is in the form of a bioreactor. The bioreactor is suited, but not limited for expansion or growth of semi-adherent or non-adherent cells. Examples of such cells include antigen-specific immune cells. The device 10 comprises a first chamber 12, which may also be referred to as a cell chamber throughout the description, and a second chamber 14, which may also be referred to as a medium chamber throughout the description. As illustrated, the first chamber 12 is arranged to be positioned on top of the second chamber 14. The first chamber 12 may be a shaped and dimensioned as a ubiquitous cell culture flask with a surface of the cell culture flask removed in a manner so as to facilitate fluid communication with the second chamber 14. The second chamber 14 may be a rectangular plate having similar longitudinal dimensions as the first chamber 12. The second chamber 14 in turn has a surface removed in a manner so as to facilitate fluid communication with the first chamber 12.

In some embodiments, the first chamber 12 and/or the second chamber 14 may be formed in part or as a whole from biocompatible materials such as Polystyrene, Polycarbonate, Polyethylene Terephthalate, Poly (methyl methacrylate), Cyclic Olefin Copolymer, or any other biocompatible material as known to a skilled person.

A semi-permeable membrane 16 is arranged to be positioned at the interface between the removed surface of the first chamber 12 and the removed surface of the second chamber 14. The semi-permeable membrane 16 may be supported by a membrane support 18, such as a grid. The grid 18 is arranged in a manner so as to provide structural rigidity to the semi-permeable membrane 16 but does not compromise the permeability of the semi-permeable membrane 16. In addition to the provision of structural rigidity, the membrane support 18 may surround the semi-permeable membrane to form a protective cover so as to protect the semi-permeable membrane 16 from sagging or being strained or torn during cell culture and piston (described later) movement.

It is appreciable that grid 18 is porous and can be of a variety of shapes and sizes as long as it can support and protect the semi-permeable membrane without affecting the permeability of the semi-permeable membrane 16. The semi-permeable membrane 16 operates to facilitate nutrient and waste exchange between the first chamber 12 and second chamber 14.

The semi-permeable membrane 16 can include pores that are arranged uniformly. The pores are micro-sized in the range of 0.1 to 2 micro-meters (μm).

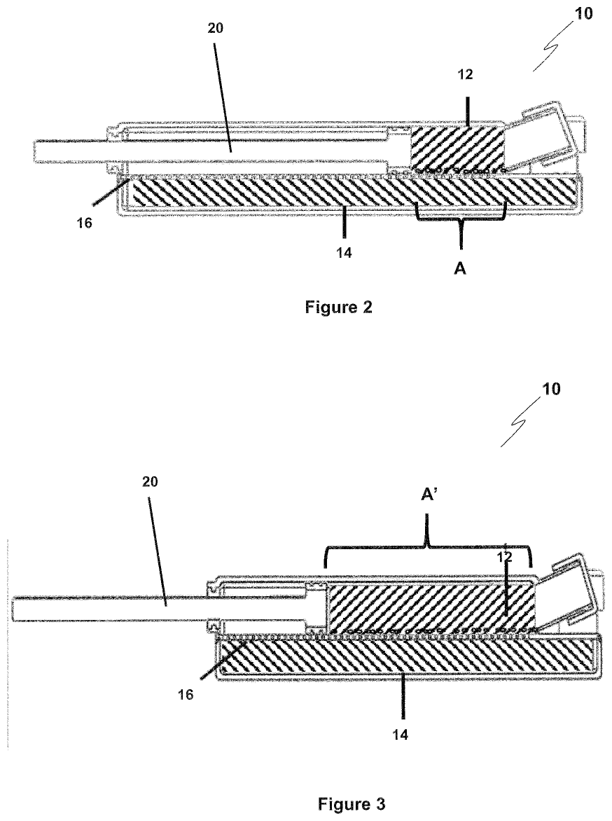

A piston 20 is disposed within the first chamber 12 for defining an area A for cell culture within the first chamber 12. The piston 20 comprises a piston head 22 and a rod 24. Piston head 22 is shaped and dimensioned to form a wall across a width of the first chamber 12 in a manner such as to define an area within the first chamber 12 for cell culture, cell growth and/or expansion. Rod 24 is attached to the piston head 22 for a user to move the piston head 22 along an axis, such as a longitudinal axis within the first chamber 12 and in doing so, adjust the area A within the first chamber 12. In use, the piston head 22 can be moved away from a wall of the first chamber 12 via a pulling action to increase the culture area/volume and bring cell density down to optimum range (see FIGS. 2 and 3 with respective cell culture areas increased from A to A′). It is appreciable that the movement (e.g. pulling action) is to be performed with a controlled speed so that the culture cells are not exposed to sudden pressure fluctuation which may affect cell growth and/or create unnecessary turbulence within the first chamber 12.

In some embodiments, the stopping position of the piston 20 at different stages of cell growth can be guided by graduated markings of cell culture area on the cell chamber 12.

A portion is cut out from a side 12a of the first chamber 12 to form a hole. The hole is adapted for the rod 24 to be inserted.

To minimize leak of contents, a sealant, such as bio-compatible rubber compounds 26 may surround the piston head 22. An example of a suitable rubber compound may be polydimethylsiloxane. The rubber compound assists to maintain water tightness within the cell culture area and prevent cells from leaking out of the cell culture area. Another rubber compound 28 may surround the inner circumference of the hole formed on side 12a to secure the rod in place and to avoid contamination from the environment.

An opening 30 is formed on the first chamber 12 and positioned for direct access to the area for cell culture. The opening 30 can be used for cell seeding, medium allocation, cell resuspension, sampling and cell harvesting. A filter cap 32 is used to cover the opening 30 in use. Filter cap 32 comprises sterile filter which allows filtered air into the cell chamber to facilitate continuous gaseous supply to the cell.

The opening 30 may be in the form of a protruding port having inner diameter between 2.5 millimeters (mm) to 20 mm to allow a variety of filter cap sizes to fit thereon. The port 30 may comprises screw threads or snap-fit engagement means for engaging with common filter cap 32. The opening 30 can be shaped and dimensioned to allow the use of serological pipettes or other pipetting devices to gently flush the first chamber 12. This ensures cells are properly spread out throughout the culture area.

As illustrated in particular in FIG. 2, in order to facilitate the use of serological pipettes or other pipetting devices to gently flush the first chamber 12, the opening or port 30 is angled or inclined with respect to the longitudinal axis of the first chamber 12. Such an arrangement facilitates gentle flushing of cell chamber to distribute cells throughout expanded culture area. Further, the gradually narrowed mouth of the first chamber 12 facilitates the use of serological pipettes or other pipetting devices to gently flush the cell chamber. This ensures cells are properly spread out throughout the culture area/volume. Cell harvesting and sampling can therefore be easily performed through the cell chamber port.

One or more openings 34 may be formed on the first chamber 12 distal from the area for cell culture. The openings 34 may be filtered. As illustrated, the one or more openings 34 may be formed at one or more corners of the first chamber 12 proximate the side 12a where the rod 24 protrude therefrom. The openings 34 facilitate the aspiration/drawing of air upon the sliding of the piston 20 along the length of the first chamber 12. They also facilitate gaseous exchange between the environment and the cell culture space. The number of filtered holes can be varied depending on the operational needs. In general, more filtered holes will allow air to be aspired quicker.

The second chamber 14 is operably arranged to be below the first chamber 12 for containing medium to facilitate the desired cell culture within the first chamber 12. Second chamber 14 comprises one or more openings 40 to facilitate gaseous exchange between the ambient environment and the second chamber 14. The openings 20 may be fitted with sterile filtered cap 42 (similar in function with filter cap 32) to facilitate gaseous exchange to medium chamber. The opening 40 may be in the form of a protruding port having inner diameter between 2.5 mm to 20 mm to allow a variety of filter cap sizes. The port may comprises screw threads or snap-fit engagement means for engaging with common filter cap 42.

The device 10 will be described in the context of its use for culturing semi-adherent or non-adherent cells. In operation, the first chamber 12 is arranged above the second chamber 14.

At the outset, suitable culture medium for the desired cell culture may be placed in the second chamber 14 via the ports 40. The culture medium may be filled until medium level is at a suitable level above the semi-permeable membrane 16. This provides just enough medium to facilitate cell growth/expansion without ‘flooding’ the first chamber 12. The suitable level should not hinder gaseous exchange with the cells. It is appreciable that depending on cell type, number of cells, the suitable level may change.

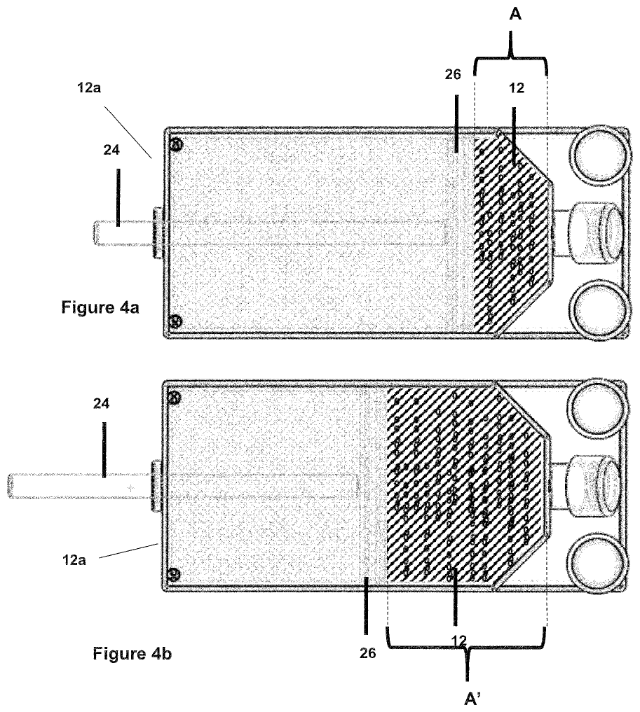

The specific cells and medium is next introduced (seeded) into the cell chamber 12 through the port 30 of cell chamber until the cell solution fully covers the cell growth area A as confined by the piston head 22 (see FIG. 2 and FIG. 4a). Cells is placed on the semi-permeable membrane 16 to receive maximum exposure to the medium.

Essential growth factors are next added through the ports 42 to medium chamber 14.

The sterile filtered vented caps 32, 42 are next attached to the respective ports, for culture.

In case the medium needs to be replaced or changed, partial removal and/or addition of medium in medium chamber 14 via the ports 40 of the medium chamber 14 may be done without affecting the cell chamber 12. The semi-permeable membrane forms a protective film against inadvertent contamination during replacement or addition of culture medium.

In order to maintain optimum cell density, the piston 20 may be moved via pulling the rod 24 to increase the area or volume (from A to A′) for cell growth and expansion (see FIG. 3 and FIG. 4b). As the rod 24 is pulled, air may be drawn out of the cell chamber 12 via openings 34 which function as air vents to facilitate the aspiration of air out of the cell chamber 12. The rubber sealants surrounding the piston head 22 and first chamber 12 facilitate smooth sliding movement of the piston 20.

The device 10 allows cell growth and expansion to be supported on the semi-permeable membrane in the cell chamber 12 without the need to transfer to other vessels/devices.

In some embodiments, stimulating cells or feeder cells can be periodically added into the cell chamber 12 through the port 30 of the cell chamber 12 to stimulate the targeted cells. In some embodiments, growth factors and other reagents can be added into the cell chamber 12 and medium chamber 14 through respective ports 30, 40. Ports 30, 40 also allows ease of sampling of cells and medium.

In some embodiments, cultured cells can be harvested from the cell chamber 12 by gentle flushing of cell chamber 12 and aspiration through the cell chamber port 30.

In some embodiments, the device 10 may form part of an intelligent system or platform for culturing different types of semi- and non-adherent cells. For example, the system may comprise different sensors or measurement devices to obtain data relating to pH, dissolved oxygen, temperature, metabolites and cell mass. The device 10 can be fitted with feedback control mechanisms to facilitate automation (for example controlling the movement of the piston 20), thus omitting the need for manual labour, and thereby facilitating remote access to control the device 10. The platform or system may be equipped with control mechanisms such as wired or wireless control. Data collection of the cell culture process may be collected overtime via the sensors to build a database for analysis and/or data mining.

In some embodiments, the openings 30, 40 may be replaced by tubing connectors for connection (via tubings or tubes) to one or more regulators to facilitate fluid exchange, such as for providing filtered air to the first chamber 12 and/or the second chamber 14. It may be appreciable that such an arrangement is a closed system that minimizes interaction with the ambient environment and therefore reduces the likelihood of contamination.

In some embodiments (not shown), there may comprise multiple pistons for adjusting the area or volume for cell from various dimensions. For example, in addition to the piston 20, one or more additional pistons may be positioned such that they move along other axis (axes) of the first chamber 12. In an alternative embodiment, a single piston may be positioned such that the movement of the piston results in adjustment to the area/volume in more than one axis.

In some embodiments relating to the control mechanisms, one or more pumps may be positioned to transmit liquid in and out of the device 10, and the device 10 may be controlled to be moved as a whole (for example to promote mixing of the cell culture with the culture medium). The one or more regulators can be physically connected to the device 10. There may further comprise additional regulators for regulating various parameters of the ambient environment, such as oxygen O₂ level, carbon dioxide CO₂ level, temperature, light around the vessel etc. These additional regulators may not need to be arranged in physical contact with the vessel.

It is appreciable that the overall control system may be “closed” in the sense that cultured cells are not directly exposed to the ambient air throughout the whole culture or cell growth process. The cells can be kept in a sterile environment with suitable growth environment control through the bioreactor and control system.

In addition, monitoring system can be added and integrated with the control system for closed-loop control. There could be additional sensors or monitoring devices which can be suitably positioned to obtain data relating to parameters such as cell morphology, cell number, medium composition, metabolites and specific cell functions.

The described features of the device 10 is apparent in at least the following ways.

It is appreciable that the device 10 reduces the time for cell culture of semi-adherent and non-adherent cells by omitting the need for centrifugation, decanting, and resuspension. Instead, only steps of medium extraction and addition is required.

The device 10 is able to maintain constant cell density with expandable culture area/volume, minimizes flow shear during cell culture, supports efficient gas exchange, and avoids the need of transferring cells across multiple discrete vessels for semi- and non-adherent cell expansion through innovative bioreactor design.

Cells are allocated and maintained inside the cell chamber. Cell settling (for non-adherent cells) and attachment (for semi-adherent cells) are facilitated by the porous semi-permeable membrane between the cell chamber and the medium chamber. The membrane encourages cell motility and interaction.

The arrangement of the cell chamber 12 on top of the medium chamber 14, separated by a semi-permeable membrane allows continuous molecule exchange across the two chambers. Waste materials produced by cells diffuse through the semi-permeable membrane from the cell chamber into the medium chamber. Nutrients diffuse from the medium chamber into cell chamber in the reverse manner.

Gaseous exchange in both cell and medium chambers may be achieved via the openings 30, 40 (with filtered caps 32, 42) which allows filtered air into the cell chamber to facilitate continuous gaseous supply.

Cells to be cultured are maintained in minimum amounts of medium on top of the semi-permeable membrane in the cell chamber. The low medium height above the cells facilitates efficient direct gaseous exchange between the cells and the air space above it.

The medium chamber 14 comprises filtered venting ports 42 which facilitate continuous gaseous exchange between medium chamber and the environment.

Cell density control can be achieved without transfer to a bigger vessel. The piston 20 can move inside the cell chamber to gradually and continuously adjust the culture area/volume for the expanding cells and bring down cell density when cell number increases, helping keep cell density within an optimum range for the specific cell type.

The device 10 supports expandable culture area/volume in the same culture vessel, thus cell transfer to larger/multiple culture flasks when cell number increases is no longer necessary. Bulk culture medium can be placed in the medium chamber 14 to support continuous cell culture for relatively long culture periods. Partial medium change can be performed in the medium chamber through the medium chamber port when nutrients run low. As cell chamber 12 is not touched during medium change, cell secreted growth factors and proteins inside the cell chamber can be better retained compared to existing systems.

The device 10 enables medium exchange and sampling to be performed in the medium chamber 14 without directly touching the cells inside the cell chamber 12. The flow shear and disturbance to cells due to medium manipulation can thus be minimized.

By separating the cell chamber 12 and medium chamber 14, the device 10 also maintains the cell culture at a low culture medium volume so as to reduce rocking and wave motion experienced by the cells, and to improve gaseous exchange to cells, while maintaining continuous and sufficient nutrient supply.

In summary, it is appreciable that the aforementioned features support continuous cell culture with reduced/minimized cell growth disruption. The device 10 enables in-situ culture area expansion and cell density regulation without the need of transferring cells to larger/multiple vessels; change of medium without direct contact with the cells; good shear flow control around the cells; enhanced gaseous exchange to cells; and continuous cell culture process. This helps reduce user-handling; cell exposure to the environment; and reduces contamination risk. The reduction in user-handling and enhanced user-friendliness is illustrated in Table 1 for the major steps required in clinical culture of immune cells.

Table 1 compares the difference in protocol for major steps between a cell culture method/system with 24-well plate device and the device 10, using antigen-specific cytotoxic T cells and the cells to be grown. (PBMC: peripheral blood mononucleocyte; IL-2: Interleukin-2)

TABLE 1

| No. | Steps | 24-well plate | Invention |
|---|---|---|---|
| 1 | Initial seeding | Seed PBMC suspension | Seed cell suspension through |
| | | into 24 well plates | the seeding port into the first |
| | | Requires one round of | chamber 12. |
| | | pipetting for each well | Requires only one round of |
| | | (multiple aliquots) | pipetting in total (one aliquot) |
| 2 | Medium change | Transfer 50% media per | NO TRANSFER OF MEDIA |
| | | well into centrifuge tube | OUT |
| | | gently to avoid agitating | |
| | | cells (multiple aliquots) | |
| | | Centrifuge -10 mins | NO CENTRIFUGATION |
| | | Re-suspend cells in same | NO RESUSPENSION OF |
| | | volume of fresh media | CELLS |
| 3 | Cell passaging - | Flush each well to fully | Flush the cell culture |
| | cell detachment | dislodge adhered cell | chamber (first chamber 12) to |
| | | clumps (multiple aliquots) | fully dislodge adhered cell |
| | | Pool the dislodged cells | clumps (one aliquot) |
| | | from each well into | NO POOLING OF CELLS |
| | | centrifuge tube | Do cell count to estimate |
| | | Do cell count to estimate | required number of |
| | | required number of | stimulating cells |
| | | stimulating cells | |
| | Cell passaging - | Dilute cells in fresh media | Pull the plunger (and hence |
| | dilution | to attain suitable cell | activate movement of the |
| | | density (one aliquot) | piston 20) in cell culture |
| | | | chamber to the suitable |
| | | | culture position to attain |
| | | | suitable cell density (one |
| | | | aliquot) |
| | Cell re-seeding | Aliquot cell solution into | NO CELL RESEEDING |
| | | each well of 24-well plate | |
| | | (multiple aliquots) | |
| 4 | Addition of | Add IL-2 to the cell | Add IL-2 to medium chamber |
| | growth factors | solution to attain suitable | (second chamber 14) to attain |
| | | IL2 concentration (one | suitable IL2 concentration |
| | | aliquot) | (one aliquot) |
| 5 | Stimulation | Add stimulating cell | Add stimulating cells to cell |
| | | solution into each well | culture chamber |
| | | (multiple aliquots) | (one aliquot) |
| 6 | Final harvest | Pipette from each well and | Pipette from harvesting port |
| | | pool cells from each well | and pool cells in one go (one |
| | | separately (multiple | aliquot) |
| | | aliquots) | |

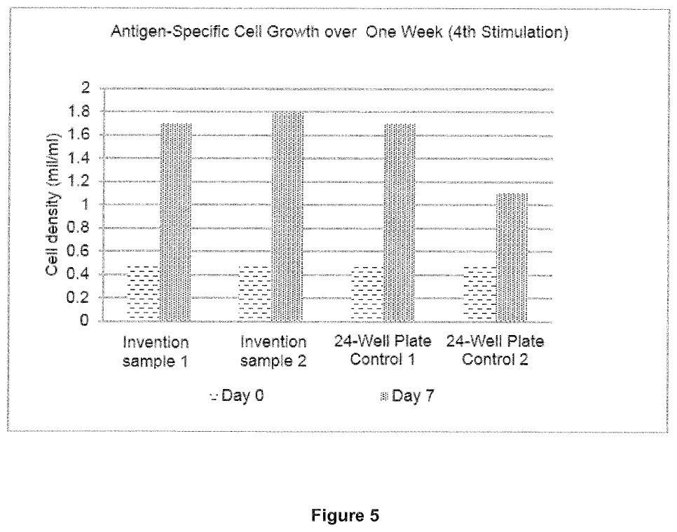

An experiment was conducted to compare the cell growth of antigen-specific T cells in the invented bioreactor with that in 24-well plate (control). As illustrated in FIG. 5, the cell density in the prototype over one week period (the 4th stimulation) is comparable to that in 24-well plate. Hence comparable growth was achieved with reduced contamination risk and user-handling effort with the invention.

While the piston 20 is described in the context of being pulled (via the rod 24) to increase the area or volume for cell growth, it is of course appreciable that the rod 24 may be pushed to decrease the area (under resetting procedures or processes).

It is appreciable that while the moveable wall defining the cell culture area/region is described as part of a piston, other types of mechanisms for moving the wall are possible.

In another embodiment (not shown), the device 10 may be modified into a single chamber device. In such an embodiment, the first chamber 12 and second chamber 14 may be combined into a single chamber without the need for a semi-permeable membrane as a divider between the two chambers. It is appreciable that the single chamber will be used to contain both cells (for culture) and the medium. Both the medium and cells can be introduced to the single chamber. The piston 20 will be utilized to adjust the area/volume for containing cells for growth.

In another embodiment (not shown), the first chamber 12 which may be a ubiquitous cell culture flask may have a surface removed and fitted with a piston. It is appreciable that the second chamber 14 and/or the semi-permeable membrane may not be necessary and the cell culture and medium may be placed in the first chamber 12, with the piston 20 adjusting the area/volume for cell growth. It is further appreciable that the first chamber 12 may contain the necessary ports and filter caps to facilitate introduction of cells and/or medium as well as provide a closed environment or closed system where the area for containing cells and culture medium is isolated from the ambient environment so as to minimize interaction with the ambient environment. In some embodiments, the first chamber 12 may be connected to the one or more regulators as described to achieve the closed environment or closed system.

It should be further appreciated by the person skilled in the art that variations and combinations of features described above, not being alternatives or substitutes, may be combined to form yet further embodiments falling within the intended scope of the invention.

1. A device for cell culture, the device comprising
a first chamber having an area for containing cells for growth;
a second chamber for containing culture medium;
a semi-permeable membrane forming at least part of a divider between the first chamber and the second chamber;
wherein the area for containing cells for growth is adjustable via a moveable wall disposed within the first chamber.

2. The device of claim 1, wherein the semi-permeable membrane comprises uniform micro-sized pores.

3. The device of claim 2, wherein the diameter of each micro-sized pore is between 0.1 to 2 micro-meters (μm).

4. The device of claim 1, wherein the semi-permeable membrane is supported or covered by a porous grid.

5. The device of claim 1, wherein the moveable wall is part of a piston and the first chamber comprises a port to receive the piston.

6. The device of claim 5, wherein the piston is operably movable along a longitudinal axis of the first chamber.

7. The device of claim 6, wherein the port is coated with a sealant.

8. The device of claim 1, wherein the moveable wall is coated with a sealant.

9. The device of claim 7, wherein the sealant is a bio-compatible rubber.

10. The device of claim 1, wherein the second chamber comprises at least one filtered opening shaped and dimensioned for gaseous exchange with the environment.

11. The device of claim 1, wherein the first chamber comprises at least one filtered opening shaped and dimensioned to receive cells.

12. The device of claim 1, wherein the first chamber comprises at least one opening located distal from the area for containing cells for growth.

13. A method for culturing cells, comprising the steps of:
providing a first chamber, the first chamber having an area for containing cells for growth;
providing a second chamber for containing culture medium;
wherein the first chamber and the second chamber are separated using a semi-permeable membrane, the semi-permeable membrane forming part of a divider between the first chamber and the second chamber; and
wherein the area for containing cells for growth is adjustable via a moveable wall disposed within the first chamber.

14. A method for culturing semi-adherent and non-adherent cells, the method comprising:
providing the device of claim 1;
introducing culture medium into the second chamber of the device until the medium level is at a suitable level above the semi-permeable membrane of the device.

15. A system for cell culture comprising
the device of claim 1;
a control mechanism;
wherein the control mechanism is operable to adjust the area for containing cells for growth via moving the moveable wall.

16. The system of claim 15, wherein the control mechanism comprises a regulator operable to be connected to the device for fluid exchange, the regulator arranged to form a closed system with the device.

17. (canceled)

18. The device of claim 3, wherein the semi-permeable membrane is supported or covered by a porous grid.

19. The device of claim 6, wherein the moveable wall is coated with a sealant.

20. The device of claim 7, wherein the moveable wall is coated with a sealant.

21. The device of claim 8, wherein the sealant is a bio-compatible rubber.
